(19) United States
(12) Patent Application Publication
(10) Pub. No.: US 2022/0126099 A1
(43) Pub. Date: Apr. 28, 2022

(54) PHYSIOLOGIC SIGNAL TRANSMITTER AND RECEIVER DEVICE

(71) Applicant: CEREGATE GMBH, Hamburg (DE)

(72) Inventors: BÁLINT VÁRKUTI, München (DE); SAMAN HAGH GOOIE, Hamburg (DE)

(21) Appl. No.: 17/505,966

(22) Filed: Oct. 20, 2021

(30) Foreign Application Priority Data
Oct. 23, 2020 (DE) 102020213417.0

(51) Int. Cl. A61N 1/36

(52) U.S. Cl. CPC A61N 1/36139

(57) ABSTRACT

A physiologic signal transmission system for an individual includes a physiologic signal transmitter and receiver device. The physiologic transmitter device includes a first receiver configured to obtain sensor signals monitoring physiologic states of the individual; a first processor configured to determine stimulation signals based on the obtained sensor signals, where the stimulation signals encode instructions to modulate functions of a target organ of the individual; and a stimulation device configured to apply the determined stimulation signals to a physiologic system or structure of the individual. The physiologic signal receiver device includes a second receiver configured to receive the stimulation signals from the stimulation device, a second processor configured to decode the encoded instructions from the stimulation signals, and an effector device configured to affect or modulate the function of the target organ based on the decoded instructions to correct or alleviate the monitored physiologic states of the individual.

### 1. PRIORITY CLAIM

This application claims priority to German Patent Application number DE 102020213417.0, titled “Physiologic Signal Transmitter and Receiver Device” and filed on Oct. 23, 2020, which is hereby incorporated by reference in its entirety, as though fully and completely set forth herein.

### 2. TECHNICAL FIELD

The present invention relates to physiologic signal transmitter and receiver devices, physiologic signal transmission systems and computer programs that may be used for transmitting information relating to a physiologic or mental state of an individual via artificial physiologic excitations propagating within a physiologic system or structure of the body of the individual.

### 3. TECHNICAL BACKGROUND

Homeostasis of a living body refers to the state of steady internal, physical, and chemical conditions maintained by the body. Homeostasis ensures optimal functioning for the organism and includes many physiologic variables, such as body temperature, fluid balance, hormone and neurotransmitter levels, etc. being kept within certain pre-set limits (homeostatic range). Other variables may include the pH of extracellular fluid, the concentrations of sodium, potassium and calcium ions, and blood sugar levels, and these may be regulated despite changes of the environment, diet, or level of activity of the living body. In healthy individuals each of these variables is controlled by one or more homeostatic mechanisms mainly evolved as physiologic feedback loops, which together maintain life.

Many homeostatic control mechanisms comprise interdependent components for the variable being regulated such as a physiologic receptor system or structure, a physiologic control system or structure and/or a physiologic effector system or structure. For instance, the receptor system or structure functions as a sensing component that monitors and responds to changes in the environment, either external or internal. Such receptor systems or structures may include thermoreceptors, mechanoreceptors and chemoreceptors etc.

As an example, consider blood sugar concentration regulation in humans and mammals. In mammals, the primary sensors for blood sugar levels are the beta cells of the pancreatic islets. The beta cells respond to a rise in the blood sugar level by secreting insulin into the blood, and simultaneously inhibiting their neighboring alpha cells from secreting glucagon into the blood. This combination (high blood insulin levels and low glucagon levels) act on effector organs and tissues, such as the liver, fat cells and muscle cells. The liver is inhibited from producing glucose, taking it up instead, and converting it to glycogen and triglycerides.

Several medical conditions may interfere or even interrupt such homeostatic control mechanisms. For instance, insulin and/or glucagon production and/or transmission to the respective effector organ may be impaired. Further, physiologic sugar concentration chemoreceptors may also be impaired.

To treat such conditions, implantable drug delivery systems and implantable chemosensors have been developed.

The following prior art systems may be relevant for characterizing the technological background of the present invention:

US 2017/0258370 relates to a system for provoking gait disorders usable for diagnosing and treatment. For instance, displays of situations calculated to cause freezing of gait are presented to a subject, optionally using virtual reality displays. Optionally incipit freezing of gait is identified using changes in gait parameters and used to guide attempts at causing freezing of gait. Based thereon a portable device can be configured to detect incipit freezing of gait events and generate a corrective signal to the subject.

U.S. Pat. No. 9,008,762 relates to a system that computes a cardiac-based metric based upon characteristics of a subject's cardiac function. For instance, the end of a mechanical systole is identified for each of a plurality of cardiac cycles of a subject, based upon an acoustical vibration associated with closure of an aortic valve during the cardiac cycle. The end of an electrical systole of an ECG signal for each cardiac cycle is also identified. Based thereon an electronic device can be constructed that comprises an input circuit and a computer circuit configured for receiving an electrical signal representative of an ECG from a subject and to identify a plurality of cardiac cycles in the electrical signal. Such systems are implemented to address alterations that can impact cardiac function and arrhythmic risk indicative of the changes that occur at the cellular level, which vary with time, stress, and other stimuli.

U.S. Pat. No. 7,785,249 relates to an apparatus for relieving stress using biofeedback techniques used according to a specified regimen to enable a user to achieve a relaxed state. Such an apparatus may comprise a sensor wirelessly connected to a CPU, which processes signals from the sensor to produce a visual display and/or auditory display that is representative of the relaxation state of the user.

U.S. Pat. No. 8,983,591 relates to an apparatus for detecting seizures with motor manifestations may comprise one or more electromyography (EMG) electrodes capable of providing an EMG signal substantially representing seizure-related muscle activity and a processor configured to receive the EMG signal, process the EMG signal to determine whether a seizure may be occurring, and generate an alert if a seizure is determined to be occurring based on the EMG signal.

Similarly, U.S. Pat. No. 10,543,359 relates to a medical system that implements a seizure detection algorithm to detect a seizure based on a first patient parameter. The medical system monitors a second patient parameter to adjust the seizure detection algorithm. For example, the medical system may determine a first patient parameter characteristic indicative of the target seizure detected based on the second patient parameter and store the first patient parameter characteristic as part of the seizure detection algorithm. In some examples, the first patient parameter is an electrical brain signal and the second patient parameter is patient activity. A similar system is also discussed in US 2010/0168603.

Further, U.S. Pat. No. 7,269,455 relates to a system for the detection and prevention of epileptic seizures utilizing bioelectric signals to assess a seizure profile and an adaptive control system for neurofeedback therapy. The system provides the detection of changes in the non-linear dynamics of brain electrical activities to characterize and differentiate individual susceptibility to seizure onset, predict the occurrence of a seizure episode, and initiate neurofeedback training to prevent the attack.

In this context, the earlier patent applications DE 10 2019 202 666 and US 2020/0269049 by the applicant are also relevant for specifying the technological background of the present invention.

Moreover, the article “Electroceuticals” by Geoffrey Ling and Corinna E. Lathan published in Scientific American on Sep. 14, 2018 discusses a electroceutical communication system employing natural orthodromic nerve conduction properties of neural fibers within the body for closed-loop control.

Further, US 2010/0305437 relates to a system for generating a mechanical signal in a mammal, the mechanical signal having a frequency no more than 50,000 Hz, and for transmitting the mechanical signal through the musculoskeletal system in the mammal, and sensing the mechanical signal from the musculoskeletal system. Such system can for instance be used for drug delivery by generating a mechanical signal internal or external to a mammal, transmitting the signal through the musculoskeletal system of the mammal, detecting the mechanical signal, and delivering the drug in response to the mechanical signal.

Similarly, U.S. Pat. No. 6,754,472 relates to an apparatus for distributing power and data to devices coupled to the human body. The human body is used as a conductive medium, e.g., a bus, over which power and/or data is distributed.

EP 2 208 458 relates to a network that has two different network nodes connected with a body of a patient. The two network nodes have a medical function such as diagnostic function and medication function. The network nodes are designed to directly communicate with one another via the body of the patient and exchange data and/or instructions. The network nodes include a temperature sensor, blood pressure sensor, sensor for detecting glucose, lactate, carbon dioxide, boric acid and metaboric acid and another sensor for detecting bodily functions i.e. kidney function.

Further, related prior art is provided by U.S. Pat. No. 9,812,788 and US 2006/0243288.

The review article “A Review on Human Body Communication: Signal Propagation Model, Communication Performance, and Experimental Issues” published in Hindawi Wireless Communications and Mobile Computing, Volume 2017, Article ID 5842310 provides a recent overview on human body communication systems.

### 4. SUMMARY OF THE INVENTION

The prior art systems and devices discussed above exhibit various deficiencies.

For instance, several of the discussed prior art systems require implantation of dedicated stimulation devices such as dedicated cortex stimulation electrodes via invasive surgical procedures that may not be safe and/or not yet fully approved for widespread clinical use.

Moreover, the available systems for monitoring or detecting a deteriorating physiologic or mental state of an individual may not be calibrated for individual patients and thus lack the capability to perform patient specific device functioning optimization. Moreover, the way monitoring is executed and/or how the individual may be informed about the result of the monitoring may be inefficient, inconvenient and/or unreliable.

Moreover, several of the prior art systems use bulk body tissue such as bones, fat tissue and/or bulk muscle tissue to transmit non-physiologic signals in an undirected way through the body of an individual. This approach may be harmful to the individual, easily affected by external interference, and/or require substantial transmit power due to signal dampening within the respective body tissue.

Accordingly, treating deteriorating homeostatic mechanisms and similar medial conditions with prior art devices and systems may not always lead to satisfying results.

It is thus a problem underlying the present invention to overcome such and similar deficiencies of previous technologies. The above-mentioned problems are at least partially solved by a physiologic signal transmitter device of and a physiologic signal receiver device as well as by a corresponding computer program, according to some embodiments.

Some embodiments are directed toward novel signal transmission devices and systems that can be used together with implantable drug delivery systems and implantable chemosensors to significantly improve their performance. Some embodiments may also be used in conjunction with systems and devices for monitoring of neurological, psychological, or physiological states of an individual.

Specifically, some embodiments provide a physiologic signal transmitter device for an individual, comprising a receiver (sometimes referred to as a receiver module) configured to obtain one or more sensor signals monitoring one or more physiologic and/or mental states of the individual, a processor (sometimes referred to as a processing module) operably connected to the receiver module and configured to determine one or more stimulation signals based at least in part on the obtained one or more sensor signals and a stimulation device (sometimes referred to as a stimulation module) operably connected to the processing module and configured to apply the determined stimulation signals to a physiologic system or structure of the individual via a physiologic stimulation device of the individual. The one or more stimulation signals are configured to elicit one or more artificial physiologic excitations propagating along the physiologic system or structure of the individual and the one or more artificial physiologic excitations encode information about the monitored one or more physiologic and/or mental states of the individual.

In this context and for the remaining part of this application the term “physiologic and/or mental state” is to be understood such that it does not cover a behavioral state (e.g., a movement) of the individual. Thus, the above-mentioned sensor signals do not monitor movement states or behavioral states of the individual.

In essence, embodiments herein enable implementation of a novel physiologic signal transmission system based on artificial physiologic excitations of a natural physiologic system or structure of an individual. In contrast to several of the prior art systems, this approach is not based on transmitting non-physiologic signals via bulk body tissue such as ultrasound signals or non-physiologic electrical signals.

For instance, the physiologic system or structure used by the physiologic signal transmission system may comprise one or more of the following: a muscle fiber of the individual, a nerve fiber or neuron of the individual, or a blood vessel of the individual.

Further, the artificial physiologic excitation may comprise one or more of the following: on or more action potentials, sub-threshold electrical activity of muscle fibers of the individual, sub-threshold electric potentials of nerve fibers or neurons of the individual, and/or an artificial modulation of a natural physiologic excitation of the physiologic system or structure, such as an amplitude modulated, shape modulated and/or frequency modulated heartbeat of the individual.

By avoiding transmission of non-physiological signals through bulk body tissues, unwanted side effects and/or the otherwise inevitable damping of signal strength may substantially be reduced. Embodiments herein greatly increases the versatility, biocompatibility, directivity and power efficiency of the described systems as compared to prior art systems.

In some embodiments, the natural physiologic structure or system may project to a target organ and/or a target position within the body of the individual associated with an external or surgically implanted device of the individual.

In this way, the high degree of directivity and spatial resolution of natural physiologic structures capable of propagating natural and artificial physiologic excitations can be used to interface the physiologic signal transmission device with one or more receiver devices or to communicate information about the monitored physiologic or mental states to the cortex, as explained in more detail below.

Further, a binary code may be used to encode the transmitted information. Alternatively, or additionally part of the transmitted information may also be encoded in analog form. In this way, common modulation and coding techniques know from electrical signal transmission systems may readily be applied to the physiologic signal transmission devices and systems provided by the present invention.

In some embodiments, the one or more physiologic excitations are generated by the physiologic signal transmission device such that the normal function of the physiologic system or structure and/or of the target organ or target position is not substantially affected by the one or more physiologic excitations.

In this way, the provided physiologic signal transmission device can reliably exchange information with one or more receiver devices without affecting the normal functioning of the body of the individual and even without being sensed or perceived by the individual.

In some embodiments, the stimulation module of the physiologic signal transmission device may be configured to apply the determined stimulation signals to a neurostimulation electrode of the individual. In this case, the one or more stimulation signals may be configured to elicit one or more electrophysiologic excitations propagating in one or more nerve fibers or neurons (e.g. within the vagus nerve and/or efferent motor neurons), projecting to a target organ or target position of the individual and wherein the one or more stimulation signals may then elicit one or more electrophysiologic excitations that encode information related to the obtained one or more sensor signals.

Due to their excellent signal transmission properties, nerve fibers and in particular myelinated axons allow for highly efficient signal transmission with low power consumption and high bandwidth.

For instance, if a binary code and/or analog encoding is used for physiologic signal transmission encoding the transmitted information may be based on one or more of the following: an inter-spike interval, an excitation amplitude, a spike count within a burst, a spike frequency within a bust, an excitation duty cycle and/or an excitation waveform or pulse shape.

Further, the one or more electrophysiologic excitations may be generated such that the normal function of the one or more nerve fibers or neurons and/or of the target organ or target position is not substantially affected by the one or more electrophysiologic excitations.

For instance, the one or more electrophysiologic excitations may be generated such that they lie outside a natural frequency range, amplitude range and/or excitation signal shape range of the one or more nerve fibers or neurons projecting to the target organ or position of the individual.

Alternatively, or additionally, the one or more electrophysiologic excitations may correspond to a non-natural spiking pattern within the one or more nerve fibers or neurons projecting to the target organ or target position of the individual.

More specifically, the frequency, the amplitude and/or the signal shape of the one or more stimulation signals may be chosen such that no action potentials are elicited in the one or more nerve fibers or neurons.

Alternatively, the frequency, the amplitude and/or the signal shape of the one or more stimulation signals may be chosen such that action potentials that are elicited in the one or more nerve fibers or neurons do not activate synapses of the one or more nerve fiber or neurons that affect the function of the target organ or target position.

For example, a pulse frequency of the stimulation signals may be chosen to be larger or equal to 10 kHz, a pulse duration of the one or more stimulation signals may be chosen to be smaller or equal to a 1 μs, and/or the pulse frequency of the stimulation signals may be chosen to be substantially larger than the inverse of a refractory period of the one or more nerve fibers or neurons.

In this manner, any kind of information about the one or more monitored physiologic or mental states of the individual may be safely and reliably transmitted via the nervous system of the individual to any kind of receiver device or reception organ without interfering with the normal function of the nervous system.

Importantly, the encoding of the monitored states is implemented as physiologic signals which are then transmitted along the body's own natural neural communication pathways, namely the peripheral and/or central nervous system.

For instance, in some embodiments a blood composition sensor in the carotid artery may (instead of transmitting the sensor data outside of the body or along an implanted wire) stimulate the vagus nerve in a downstream direction with a signal train that does not change the nerve function itself but can be successfully decoded by a downstream implanted second device (the recipient; see below) to react in a pre-programmed manner, forming a within-body closed-loop system using the natural nervous system as the information signaling pathway. In some embodiments, differentiated signals transmitted via the nerve fibers do not themselves alter the target organ's (where that nerve terminates) function, but rather regulate or instruct an effector device which in turn may alter that target organ's function.

In another embodiment, the stimulation module may be configured to apply the determined one or more stimulation signals to a neurostimulation electrode of the individual, wherein the one or more stimulation signals are configured to elicit one or more electrophysiologic excitations in one or more nerve fibers or neurons of the central nervous system projecting to the sensory cortex of the individual, wherein the one or more electrophysiologic excitations are configured to elicit a sensory percept in the sensory cortex of the individual, and wherein the elicited sensory percept provides information to the individual about the one or more monitored physiologic and/or mental states of the individual.

In other words, the electrophysiologic excitations generated by the physiologic signal transmitting device may not only be transmitted to electronic receiver devices but also to the sensory cortex of the individual. Naturally, the encoding of the information must be performed such, that the sensory cortex can decode it.

For such embodiments of the present invention the processing module may further be configured to derive, based on the obtained sensor signals, a continuous or categorical metric characterizing the one or more physiologic and/or mental states of the individual, and the determined one or more stimulation signals may be configured to elicit a sensory percept in the cortex of the individual indicating a current value of the derived metric to the individual (see for example FIG. 6 below).

For instance, the one or more sensor signals may monitor a mental state of the individual related to a recurring neurological condition and the derived metric may indicate to the individual a likelihood for the neurological condition to recur.

Further, the processing module may be configured such that the determining of the one or more neural stimulation signals comprises determining one or more signal parameters of the one or more neural stimulation signals based at least in part on a determination function that maps the current value of the metric to one or more values of the one or more signal parameters.

For example, the one or more signal parameters may comprise one or more of the following: one or more activated stimulation channels, a signal amplitude, a signal frequency, a signal duty cycle, a signal pulse width, a signal polarity, a signal burst frequency, a signal burst spike count and/or the determination function may comprise an activation function such as a sigmoid function, a gaussian function, a rectified linear function, a logistic function, a hyperbolic function.

In this manner the physiologic transmitter device is enabled to derive metrics about complex physiologic or mental states of the individual, such as psychologic stress levels, arousal states, depression states, and the like.

In some embodiments, the processing module may further be configured to derive, based on the obtained one or more sensor signals, a continuous or categorical metric characterizing the one or more physiological and/or mental states of the individual, to compare a current value of the metric to a reference value for the metric and, in response to determining that the current value of the metric has exceeded the reference value determine a stimulation signal that is configured to elicit a sensory percept indicating to the individual that the reference value was exceeded.

In this way, the physiologic signal transmitter device may be enabled to provide the individual with warning signals that the monitored physiologic or mental state is about to deteriorate and that countermeasures (e.g. drug or food intake, autogenic procedures, resting periods, etc.) are recommended.

The one or more stimulation signals may even be configured to elicit a multi-modal sensory percept in the cortex of the individual, e.g., an essentially synchronous visual and touch sensation. In this manner, the number of different types of information that can be transmitted about the monitored physiologic or mental states can substantially be increased.

In another embodiment, the present invention provides a corresponding physiologic signal receiver device for an individual, comprising a receiver module configured to obtain one or more physiologic measurement signals obtained from a physiologic measuring device or sensor monitoring the physiologic activity of one or more physiologic systems or structures projecting to a target organ or target position of the individual, a processing module operably connected to the receiver module and configured to extract transmitted information encoded in a subset of the obtained physiologic measurement signals, wherein the extracted transmitted information is related to one or more sensor signals monitoring one or more physiologic and/or mental states of the individual.

Such a physiologic signal receiver device may also comprise (or communicate with) one or more effector modules (or devices) configured to affect or modulate the function of the target organ or target position of the individual and/or a non-transitory computer-readable memory medium (sometimes referred to as a memory module) storing predefined signal characteristics that are used for extracting the transmitted information from the obtained physiologic measurement signals and/or a stimulation module configured for applying a blocking stimulation to the one or more natural physiologic structures blocking or canceling the propagation of an artificial physiologic excitation encoding the extracted information downstream of the physiologic signal receiver device.

For instance, the effector modules (or devices) may comprise one or more of the following: an electrostimulation module, a drug administration module, a heating and/or cooling module, a light emission module, an artificial synapse and/or a vibration or ultrasonic effector module.

Based on the specifications provided above, the present invention also provides a physiologic signal transmission system for an individual, comprising one or more of the above discussed physiologic transmitter devices and one or more of the physiologic signal receiver devices discussed above, wherein at least a subset of the physiologic systems or structures stimulated by the one or more physiologic signal transmitter devices are monitored, at least indirectly, by the one or more physiologic signal receiver devices.

In this manner, various types of present or future electroceuticals (implanted or external) can easily communicate with each in a reliable, biocompatible and highly directive and thus power efficient manner.

For instance, also integrated physiologic monitoring systems can be designed that comprise such a physiologic signal transmission system and one of the above physiologic signal transmitter devices that provide information about the monitored physiologic or mental states of the individual to the sensory cortex of the individual.

The one or more sensor signals may relate to a blood pressure, a blood composition, a drug or body substance level of the individual, a stress level, and/or a neural activity level or pattern of the individual.

For instance, such sensor signals may be received from at least one of the following sensor devices: a touch sensor; an electroencephalography device; an electromyography device; a sensor device for measuring a skin conductance, a respiratory rate, an electrocardiogram, and/or a temperature; a deep brain local field potential recording device; a chemo-sensor device for measuring the concentration of a substance in a body fluid of the individual; and an electrocorticography device.

In essence, the present invention thus allows to establish a physiologic communication channel between implanted or external transmitter and receiver devices based on artificially elicited physiologic excitations (e.g., neural or muscle excitations).

### 5. SHORT DESCRIPTION OF THE FIGURES

Various aspects of the present invention are described in more detail in the following by reference to the accompanying figures. These figures show:

FIG. 1 a diagram illustrating an individual operating a stress level monitoring system using an implanted blood pressure/heart rate sensor as well as wearable external sensor;

FIG. 2 a diagram illustrating signal transmission via a nerve to send encoded information in spikes/action potentials to a receiver implanted next to the target organ;

FIG. 3 a diagram illustrating another example of signal transmission via muscle fibers to control bladder function;

FIG. 4 a functional block diagram of a physiologic signal transmitter device according to an embodiment of the present invention;

FIG. 5 a functional block diagram of a physiologic signal receiver device according to an embodiment of the present invention;

FIG. 6 illustrates how an artificial electrophysiologic stimulation signal can be used to elicit a sensory percept in the cortex of an individual encoding a metric characterizing the stress level of an individual.

### 6. DETAILED DESCRIPTION OF SOME EXEMPLARY EMBODIMENTS

In the following, some exemplary embodiments of the present invention are described in more detail, with reference to a physiologic signal transmitter and receiver device that can be interfaced with stimulation electrodes for muscle or nerve fibers. However, the present invention can also be used with any other stimulation device capable of stimulating physiologic systems or structures of an individual that can propagate artificially elicited physiologic excitations carrying information to be transmitted.

While specific feature combinations are described in the following with respect to the exemplary embodiments, it is to be understood that not all features of the discussed embodiments have to be present for realizing the invention, which is defined by the subject matter of the claims. The disclosed embodiments may be modified by combining certain features of one embodiment with one or more features of another embodiment. Specifically, the skilled person will understand that features, components and/or functional elements of one embodiment can be combined with technically compatible features, components and/or functional elements of any other embodiment of the present invention given that the resulting combination falls within the definition of the invention provided by the claims.

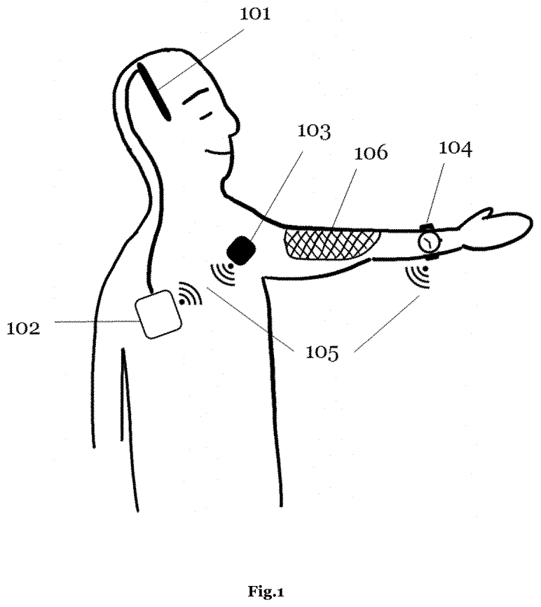

FIG. 1 depicts an individual, e.g., a patient suffering from a recurring neurological condition that may be triggered by an increased stress level. The individual has been implanted with a neuronal stimulation electrode 101 such as a spinal cord stimulation electrode or a deep brain stimulation (DBS) electrode that may have multiple independently controllable electric contacts. The individual may also use an implanted or external pulse generator & processor device 102 that may implement a physiologic signal transmission device according to an embodiment of the present invention.

For instance, pulse generator & processor device 102 may comprise a receiver module configured for receiving one or more sensor signals received from one or more sensor devices such as the implanted bio sensor 103 measuring the heart rate and/or blood pressure of the individual and/or a wearable bio sensor 104 e.g., measuring skin temperature, skin conductance, skin pressure variations, etc.

The sensor devices 103 and 104 may wirelessly transmit 105 their respective sensor signals to the pulse generator & processor device/neurostimulation device 102.

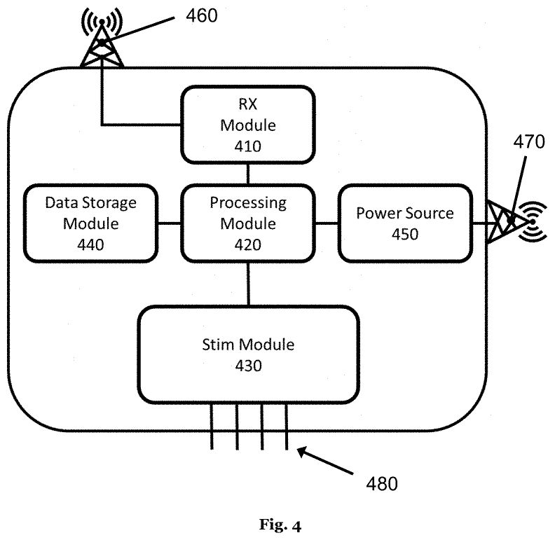

As explained in detail in section 3. “Summary of the Invention” above and with reference to FIG. 4 below, the pulse generator & processor device 102 may also comprise a processing module and a neurostimulation module for stimulating afferent sensory pathways of the central nervous system eliciting artificial sensations in the cortex of the individual as explained in detail in the applicant's earlier patent applications DE 10 2019 202 666 and US 2020/0269049 (which are both incorporated by reference in their entirety).

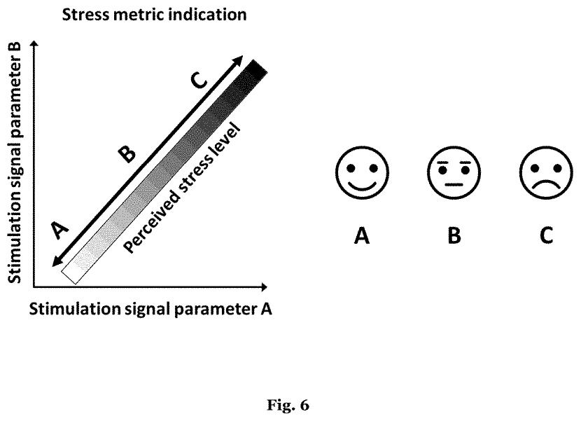

For example, the processing module may determine the applied stimulation signals based on stored specific relations based at least in part on conceptual learning data for the individual, wherein the conceptual learning data associate a plurality of information about the monitored one or more physiologic or mental states of the individual with a plurality of corresponding stimulation signals. For example, an individual may be trained to associate particular applied stimulation signals with conceptual information, and these associations may be stored in memory accessible by the processor. One specific example is shown in FIG. 6, described below, where a low frequency, short pulse width stimulation signal indicates to the individual that the individual is in a low-stress state. Conversely, a higher frequency, longer pulse width stimulation signal indicates to the individual that the individual is in a high-stress state. An individual may be trained to learn a desired association between different types of stimulation signals and different mental and/or physiological states or conceptual information.

In the illustrated embodiment, the processing module of the pulse generator & processor device 102 may for instance be configured to derive a continuous or categorical metric for a stress level experienced by the individual based on the one or more sensor signals received from the one or more implanted or wearable sensor devices 103 and 104. In some embodiments, the determined value of the metric may directly be transmitted to the individual by selecting appropriately calibrated stimulation signals (see FIG. 6 below). Alternatively, the current value off the derived metric may also be compared to a reference or threshold value and a specifically chosen stimulation signal may be determined and transmitted by the pulse generator & processor device 102 to indicate to the individual that the reference value or threshold value was crossed by the metric. In response the individual may take actions to reduce his stress level.

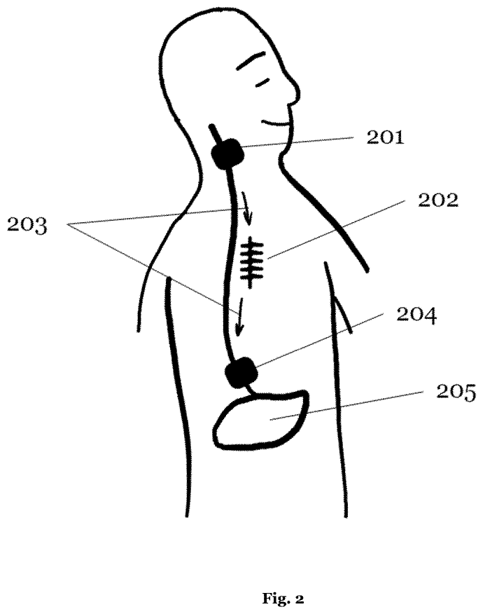

FIG. 2 depicts an individual, e.g., patient suffering from an impairment of a homeostatic feedback loop regulating the concentration of a body substance within the body of the organism. The individual has been implanted with a physiologic signal transmission system according to an embodiment of the present invention. The system comprises a physiologic signal transmitter with integrated bio sensor 201. The bio sensor may also be separated from and connected (e.g. wirelessly) to the transmitter device 201.

For instance, the sensor may be arranged in the carotid artery and may be a chemosensor monitoring the blood composition of the individual, a thermosensor, blood pressure sensor, etc. Based on the received sensor signals the physiologic signal transmitter device 201 may apply electrophysiologic stimulation signals to a nerve fiber or neuron of the individual, e.g., to the vagus nerve, in downstream direction (i.e., towards the body) via a stimulation electrode (not shown) that also may be integrated or separated from the physiologic signal transmitter device 201.

For instance, the nerve fiber or neuron may be stimulated with a signal train 202 that does not change the nerve function itself but can be successfully decoded by a downstream implanted second device (the recipient) to react in a pre-programmed manner, forming a within-body closed-loop system using the natural nervous system of the individual as the information transmission pathway 203. Advantageously, the physiologic signal transmitter 201 may utilize the human body's existing neural communication pathways to communicate with the receiver device 204 to direct modulation of the target organ 205, and this may be performed without adversely interfering with the normal biological function of the neural communication pathways. The target organ's function may be modulated to correct or alleviate the impairment detected by the integrated bio sensor of the physiologic signal transmitter. The nerve fiber may be stimulated such that artificially elicited action potentials/spikes 202 are generated. Alternatively, the stimulation parameters may also be chosen such that sub-threshold excitations propagate in downstream direction along the nerve to the receiver device. For instance, the stimulation frequency may be substantially larger than the inverse refractory period of the targeted nerve fiber or neuron.

In contrast to the embodiment of FIG. 1, in FIG. 2, conscious or even cerebral decoding and/or perception of the signal may not be performed. However, an encoding step of state-data to encoded nerve-travelling signal 202 still may occur (e.g., followed by a decoding at the recipient point(s)). It is important to differentiate that the state-data encoding signals transmitted downstream (or upstream) of the nerve do not themselves alter the target organ function (i.e., where that nerve terminates; e.g., the liver), but rather regulates or instructs a second device which in turn may then alter that target organ function.

For instance, the recipient device may be an implanted receiver and/or spike filter device 204 modulating the function of the target organ 205 (e.g., the liver, the pancreatic gland and/or the adrenal gland). Such physiologic signal receiver devices may comprise or communicate with an effector module or device that affects or modulates the function of the target organ 205.

The receiver device 204 may also comprise a receiver module for receiving electrophysiologic measuring signals monitoring the bioelectric activity of the nerve fiber or neurons stimulated by the transmitter device 201. A memory module may store predefined signal characteristics that may be used for extracting the transmitted information from the obtained physiologic measurement signals and a stimulation module may then apply a blocking stimulation to the nerve fiber or neurons for blocking or canceling the propagation of the artificial stimulation electrophysiologic signal, e.g., the receiver device may block the spikes from travelling further downstream along the nerve fiber. In this manner, the natural function of the nerve fiber downstream of the receiver device may not be affected by the information transmission between transmitter 201 and receiver 204.

Examples of effector modules or devices (not shown) comprise electrostimulation modules, drug administration modules, temperature modification modules, light emission modules (e.g., for interacting with optogenetically modified organ tissues or with light sensitive drugs such as light sensitive ion channel blockers), artificial synapses, vibration or ultrasonic effector modules, etc.

In another possible embodiment, for instance, a wearable device may receive encoded state-data travelling downstream the ulnar and medial nerves into the hand, with a high-resolution receiver device sitting on the skin and picking up the within-body data to be processed outside the body.

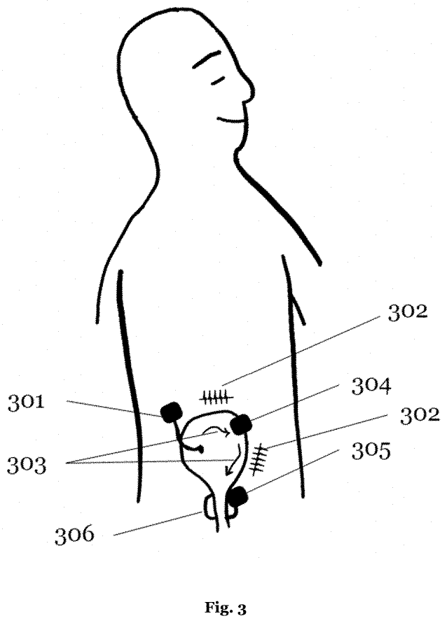

FIG. 3 illustrates a further embodiment of the present invention, wherein information is transmitted via artificial excitations of muscle fibers of the individual. More specifically, a physiologic signal transmission system provided by the present invention is used to implement a bladder function prosthesis. For instance, the urinary bladder has two important physiologic functions: storage of urine and emptying.

Storage of urine occurs at low pressure; bladder muscles relax during the filling phase. Emptying happens at high pressures and utilizes a coordinated contraction of the bladder and relaxation of the urethra muscles 306.

In some embodiments, an implanted urinary pressure bio sensor and transmitter device 301 may be attached to the body of the bladder monitoring the pressure state of the bladder. The transmitter device transmits artificial electromyography (EMG) evoked potentials encoding control information to an implanted muscle controller 304 modulating contraction function of the bladder muscles and to an implanted muscle controller 305 modulating the contraction and relaxation function of the urethra muscles 306. The control information may for instance be encoded in subthreshold muscle activity propagating through bladder body muscles.

During the bladder filling stage, the pressure sensor, for instance, detects a relatively low pressure and in response the transmitter device 301 transmits control information instructing the implanted muscle controller 304 to keep the bladder body muscles in a relaxed state and the urethra muscles 306 in a contracted state to ensure proper filling of the bladder without urine flowing out unintentionally. When the pressure sensor detects that the balder is full, the transmitter device 301 may inform the individual (e.g. electronically or via a mild vibration stimulation or via an electrophysiologic sensory excitation travelling to the sensory cortex as explained above) that the bladder should be emptied. In response, the individual may instruct the transmitter device 301 to execute bladder contraction and urethra muscle relaxation.

FIG. 4 depicts a block diagram of an exemplary physiologic signal transmitter device according to an embodiment of the present invention. In this embodiment the physiologic signal transmitter device comprises an integrated neurostimulation (“stim”) module 430 (e.g., comprising a neuronal signal generator and an output amplifier) that is connected to a plurality of output signal leads 480 that may be interfaced with a neurostimulation interface of the individual (e.g., a DBS electrode or a spinal cord stimulation electrode; not shown). The physiologic signal transmitter device further comprises a communication antenna 460 operably connected to a receiver module 410, configured for wireless communication (e.g., via NFC, BLUETOOTH™ or a similar wireless communication technology).

The receiver module 410 may be configured, for example, to receive one or more sensor signals from one or more sensors (as discussed above), indicative of one or more physiologic states of an individual (e.g., a blood composition and/or blood pressure measurement obtained from implanted or wearable chemo or pressure sensors etc.). As used herein, a “physiologic state” is meant to refer to any of a variety of physical or mental states of an individual, which may be indicated by any of a variety of biological markers. As one example, a physiologic state may refer to a mental state such as a high-stress state that is indicated by blood pressure, pulse rate, etc. Alternatively, the physiological state may refer to a physical state such as the status of the bladder (e.g., whether the bladder is full) which is indicated by a pressure sensor within the bladder. Any of a variety of other physiological states are also possible, such a state of consciousness (e.g., awake, dreaming, deep sleep, etc.) a state of a portion of the digestive system, a hormonal state, etc. The receiver module 410 may be operably connected to a data/signal processing module 420 configured to generate one or more stimulation signals and/or signal parameters (e.g., waveform, pulse shape, amplitude, frequency, burst count, burst duration etc.) for generating the one or more stimulation signals. For instance the processing module 420 may access a data storage module 440 (e.g., a non-transitory computer-readable memory medium) configured to store a plurality of relations, specific for the individual, associating a plurality of stimulation signals (or parameters used for generating a plurality of stimulation signals) with a plurality of corresponding sensory percepts associated with a plurality of respective items of information about the monitored physiologic or mental states of the individual to be transmitted to the sensory cortex of the individual.

The generated stimulation signals and/or the signal parameters may be input into the integrated neurostimulation module 430 that may be configured to process (e.g., modulate, switch, amplify, covert, rectify, multiplex, phase shift, etc.) the one or more stimulation signals generated by the processing module 420 or to generate the one or more stimulation signals based on the signal parameters provided by the processing module 420. The generated and processed stimulation signals are then output by the neurostimulation module 430 and can for instance be applied to one or more electric contacts of a neurostimulation electrode (e.g. a DBS electrode or spinal cord stimulation electrode; not shown) via output leads 480.

The illustrated physiologic signal transmitter device may also comprise a rechargeable power source 450 that, for instance may be wirelessly charged via a wireless charging interface 470.

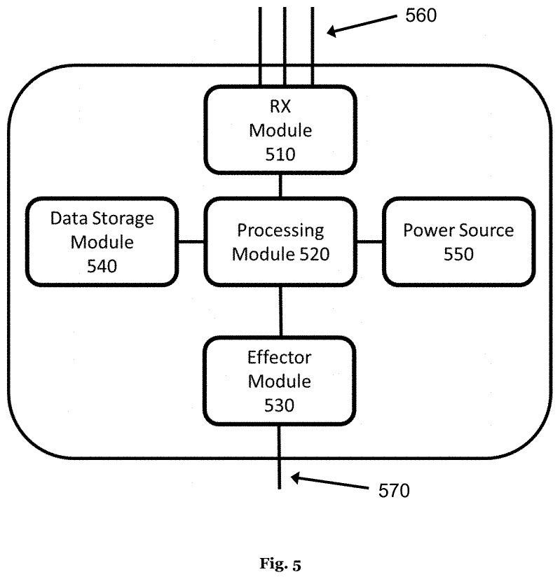

FIG. 5 depicts a block diagram of an exemplary physiologic signal receiver device according to an embodiment of the present invention. In this embodiment the physiologic signal receiver device comprises an integrated effector module 530 such as a drug administration module, an electrostimulation module, etc. The physiologic signal receiver device further comprises a receiver module 510.

The receiver module 510 may be configured, for example, to receive one or more sensor signals from one or more sensors monitoring the (electro-)physiologic activity of one or more physiologic systems or structures of the individual, such as nerve or muscle fibers (see FIG. 2 and FIG. 3 above). The receiver module 510 may be operably connected to a data/signal processing module 520 configured to extract transmitted information (e.g. transmitted by the signal transmitter device of FIG. 4) that may be encoded in a subset of the obtained physiologic measurement signals. For instance, the extracted transmitted information may be related to one or more sensor signals monitoring one or more physiologic and/or mental states of the individual as described above.

The processing module 520 may access a data storage module 540 configured to store predefined signal characteristics that can be used for extracting the transmitted information from the obtained physiologic measurement signals. For instance, the predefined signal characteristics may be used to extract non-natural spiking patterns from extracellular recordings of myelinated axons of the central or peripheral nervous system.

The processing module 520 may generate instructions for the integrated effector module 530 such as instructions to begin or cease administration of a drug or electrostimulation. The effector module 530 may also be configured to apply a blocking stimulation to the monitored physiologic structure or systems via output wire 570.

The illustrated physiologic signal receiver device may also comprise a power source 550 such as an exchangeable battery.

FIG. 6 illustrates how an artificial electrophysiologic stimulation signal can be used to elicit a sensory percept in the cortex of an individual encoding a metric characterizing the stress level of an individual, e.g., derived by the processing module 420 of the physiologic signal transmitter device of FIG. 4 based on the one or more received sensor signals.

For instance, the measurement signals of the implanted and wearable sensors discussed above with reference to FIG. 1 may be used to derive a metric quantifying the stress level experienced by an individual. The processing module may further determine a stimulation signal that is to be applied to nerve fibers or neurons of the central nervous system (e.g., the spinal cord) targeting the sensory cortex of the individual. The determined (neuro-) stimulation signal may correspond to the current value of the derived metric that is to be communicated to the individual.

As shown in FIG. 6, such a stress level metric may be encoded by a combination of stimulation signal parameters such as a pulse width, a pulse amplitude, a pulse frequency, etc. of a pulse train signal evoking specific and distinguishable sensory percepts in the sensory cortex of the individual such as different and distinguishable artificial touch sensations in the left hand of the individual. In the example shown in FIG. 6, the signal parameter A may for instance corresponds to pulse width and the signal parameter B to a stimulation frequency. In this case, the elicited sensory percept corresponding to a low frequency pulse train having a short pulse width (A) may indicate a low stress level (A) whereas a high frequency pulse train having a long pulse width (C) may evoke a different sensory percept that indicates a critical stress level (C) that may require intervention by the individual.

In some embodiments, a computer program includes instructions, executable by one or more processors, for implementing the physiologic signal transmitter devices, the physiologic signal receiver devices, and/or the systems described herein.

What is claimed is:

1. A physiologic signal transmitter device for an individual, comprising:
a receiver configured to obtain one or more sensor signals monitoring one or more physiologic states of the individual;
a processor operably connected to the receiver and configured to determine one or more stimulation signals based at least in part on the obtained one or more sensor signals; and
a stimulation device operably connected to the processor and configured to apply the determined stimulation signals to a physiologic system or structure of the individual via a physiologic stimulation device of the individual,
wherein the one or more stimulation signals are configured to elicit one or more artificial physiologic excitations propagating along the physiologic system or structure of the individual, and
wherein the one or more artificial physiologic excitations encode information about the monitored one or more physiologic states of the individual.

2. The physiologic signal transmitter device of claim 1, wherein the physiologic system or structure comprises one or more of the following:
a muscle fiber of the individual;
a nerve fiber or neuron of the individual; and
a blood vessel of the individual; and
wherein the artificial physiologic excitation comprises one or more of the following:
one or more action potentials;
sub-threshold electrical activity of muscle fibers of the individual;
sub-threshold electric potentials of nerve fibers or neurons of the individual;
an artificial modulation of a natural physiologic excitation of the physiologic system or structure, such as an amplitude modulated, shape modulated, or frequency modulated heartbeat of the individual.

3. The physiologic signal transmitter device of claim 1,
wherein the physiologic structure or system projects to a target organ or a target position within the body of the individual associated with an external or surgically implanted device of the individual.

4. The physiologic signal transmitter device of claim 1,
wherein a binary code is used to encode the transmitted information; or
wherein the transmitted information is encoded in analog form.

5. The physiologic signal transmitter device claim 4, wherein the binary code or the analog encoding is based on one or more of the following:
an inter-spike interval;
an excitation amplitude;
a spike count within a burst;
a spike frequency within a bust;
an excitation duty cycle;
an excitation waveform or pulse shape.

6. The physiologic signal transmitter device of claim 1,
wherein the one or more artificial physiologic excitations are generated such that normal function of the physiologic system or structure and of the target organ or target position is not substantially affected by the one or more artificial physiologic excitations.

7. The physiologic signal transmitter device of claim 1,
wherein the stimulation device is configured to apply the determined stimulation signals to a neurostimulation electrode of the individual,
wherein the one or more stimulation signals are configured to elicit one or more electrophysiologic excitations propagating in one or more nerve fibers or neurons projecting to a target organ or position of the individual; and
wherein the one or more stimulation signals elicit one or more electrophysiologic excitations that encode information about the monitored one or more physiologic states of the individual.

8. The physiologic signal transmitter device of claim 7,
wherein the one or more electrophysiologic excitations are generated such that the normal function of the one or more nerve fibers or neurons and of the target organ or target position is not substantially affected by the one or more electrophysiologic excitations.

9. The physiologic signal transmitter device of claim 8,
wherein the one or more electrophysiologic excitations are generated such that they lie outside a natural frequency range, amplitude range or excitation signal shape range of the one or more nerve fibers or neurons projecting to the target organ or position of the individual, or
wherein the one or more electrophysiologic excitations correspond to a non-natural spiking patter within the one or more nerve fibers or neurons projecting to the target organ or target position of the individual.

10. The physiologic signal transmitter device of claim 8,
wherein one or more of the frequency, the amplitude, and the signal shape of the one or more stimulation signals is chosen such that no action potentials are elicited in the one or more nerve fibers or neurons; or
wherein the one or more of the frequency, the amplitude, and the signal shape of the one or more stimulation signals is chosen such that action potentials that are elicited in the one or more nerve fibers or neurons do not activate synapses of the one or more nerve fiber or neurons that affect the function of the target organ or target position.

11. The physiologic signal transmitter device of claim 8,
wherein one or more of:
a pulse frequency of the stimulation signals is larger than or equal to 10 kHz;
a pulse duration of the one or more stimulation signals is smaller or equal to a 1 μs; and
a pulse frequency of the stimulation signals is substantially larger than the inverse of a refractory period of the one or more nerve fibers or neurons.

12. The physiologic signal transmitter device of claim 1,
wherein the stimulation device is configured to apply the determined one or more stimulation signals to a neurostimulation electrode of the individual,
wherein the one or more stimulation signals are configured to elicit one or more electrophysiologic excitations in one or more nerve fibers or neurons of the central nervous system projecting to the sensory cortex of the individual,
wherein the one or more electrophysiologic excitations are configured to elicit a sensory percept in the sensory cortex of the individual, and
wherein the elicited sensory percept provides information to the individual about the one or more monitored physiologic states of the individual.

13. The physiologic signal transmitter device of claim 12,
wherein the processor is further configured to derive, based on the obtained sensor signals, a continuous or categorical metric characterizing the one or more physiologic states of the individual, and
wherein the determined one or more stimulation signals are configured to elicit a sensory percept indicating a current value of the derived metric to the individual.

14. The physiologic signal transmitter device of claim 13,
wherein the processor is configured such that the determining of the one or more neural stimulation signals comprises:
determining one or more signal parameters of the one or more neural stimulation signals based at least in part on a determination function that maps the current value of the metric to one or more values of the one or more signal parameters.

15. The physiologic signal transmitter device of claim 14,
wherein the one or more signal parameters comprise one or more of the following:
one or more activated stimulation channels,
a signal amplitude,
a signal frequency,
a signal duty cycle,
a signal pulse width,
a signal polarity,
a signal burst frequency,
a signal burst spike count, and
wherein the determination function comprises an activation function, wherein the activation function comprises one or more of a sigmoid function, a gaussian function, a rectified linear function, a logistic function, and a hyperbolic function.

16. The physiologic signal transmitter device of claim 12, wherein the processor is further configured to:
derive, based on the obtained one or more sensor signals, a continuous or categorical metric characterizing the one or more physiological states of the individual;
compare a current value of the metric to a reference value for the metric; and
in response to determining that the current value of the metric has exceeded the reference value, determine a stimulation signal that is configured to elicit a sensory percept indicating to the individual that the reference value was exceeded.

17. The physiologic signal transmitter device of claim 12,
wherein the one or more stimulation signals are configured to elicit a multi-modal sensory percept in the cortex of the individual.

18. A physiologic signal receiver device for an individual, comprising:
a receiver configured to obtain one or more physiologic measurement signals obtained from a physiologic measuring device monitoring the physiologic activity of a physiologic system or structure of the individual projecting to a target organ or position of the individual;
a processor operably connected to the receiver and configured to extract transmitted information encoded in a subset of the obtained physiologic measurement signals,
wherein the extracted transmitted information is related to one or more sensor signals monitoring one or more physiologic states of the individual.

19. The physiologic signal receiver device of claim 18, further comprising:
one or more effectors configured to affect or modulate the function of the target organ or position based at least in part on the extracted transmitted information;
a non-transitory computer-readable memory medium storing predefined signal characteristics that are used for extracting the transmitted information from the obtained physiologic measurement signals; and
a stimulation device configured for applying a blocking stimulation to the physiologic structure blocking or canceling the propagation of an artificial physiologic excitation encoding the extracted information downstream of the physiologic signal receiver device.

20. The physiologic signal receiver device of claim 19, wherein the one or more effectors comprise one or more of the following:
an electrostimulation device;
a drug administration device;
a heating or cooling device;
a light emission device;
an artificial synapse;
a vibration or ultrasonic effector.

21. A physiologic signal transmission system for an individual, comprising:
a physiologic transmitter device, comprising:
a first receiver configured to obtain one or more sensor signals monitoring one or more physiologic states of the individual;
a first processor operably connected to the first receiver and configured to determine one or more stimulation signals based at least in part on the obtained one or more sensor signals, wherein the one or more stimulation signals encode instructions to modulate one or more functions of a target organ of the individual; and
a stimulation device operably connected to the first processor and configured to apply the determined stimulation signals to a physiologic system or structure of the individual,
wherein the one or more stimulation signals are configured to elicit one or more artificial physiologic excitations propagating along the physiologic system or structure of the individual; and
a physiologic signal receiver device, comprising:
a second receiver configured to receive the one or more stimulation signals from the stimulation device;
a second processor operably connected to the second receiver and configured to decode the encoded instructions from the one or more stimulation signals; and
an effector device configured to affect or modulate the function of the target organ based at least in part on the decoded instructions.

22. The physiologic signal transmission system of claim 21,
wherein affecting or modulating the one or more functions of the target organ corrects or alleviates the monitored one or more physiologic states of the individual.

23. The physiologic signal transmission system of claim 21,
wherein the one or more sensor signals relate to one or more of a blood pressure, a blood composition, a drug or body substance level, a stress level, or a neural activity pattern of the individual.

24. The physiologic signal transmission system of claim 21,
wherein the one or more sensor signals are received from one or more of the following sensor devices:
a touch sensor;
an electroencephalography device;
an electromyography device;
a sensor device for measuring one or more of a skin conductance, a respiratory rate, an electrocardiogram, and a temperature;
a deep brain local field potential recording device;
a chemo-sensor device for measuring the concentration of a substance in a body fluid of the individual; and
an electrocorticography device.
